(19) United States
(12) Patent Application Publication
(10) Pub. No.: US 2010/0323031 A1
(43) Pub. Date: Dec. 23, 2010

(54) Synergistic combination to enhance blood glucose and insulin metabolism

(75) Inventors: Daniel Elliot Clouatre, Berkeley, CA (US); Dallas Lavoe Clouatre, Berkeley, CA (US)

Correspondence Address: OLIFF & BERRIDGE, PLC, P.O. BOX 320850, ALEXANDRIA, VA, 22320-4850, US

(73) Assignee: GLYKON TECHNOLOGIES GROUP, LLC, Berkeley, CA (US)

(21) Appl. No.: 12/457,776

(22) Filed: Jun. 22, 2009

(51) Int. Cl. A61K 36/185; A61K 33/30; A61K 33/32; A61K 33/24; A61P 3/10

(52) U.S. Cl. 424/639; 424/725; 424/641; 424/655

(57) ABSTRACT

The present disclosure discloses a synergistic combination comprising (−)-hydroxycitric acid, bitter melon extract and sesame seed lignans extract to enhance blood glucose and insulin metabolism. The synergistic combination also reduces reactive hypoglycemia in those who are insulin resistant.

### TECHNICAL FIELD

The present disclosure is related to synergistic combination and its use as food and pharmaceutical compositions containing (−)-hydroxycitric acid, its salts, amides and esters in conjunction with bitter melon and sesame seed lignans to enhance blood glucose and insulin metabolism.

### BACKGROUND

Elevated and erratic blood sugar levels are components of the condition known as diabetes mellitus. This condition can be life-threatening and high glucose levels in the blood plasma (hyperglycemia) can lead to a number of chronic diabetes syndromes, for example, atherosclerosis, microangiopathy, peripheral neuropathy, kidney disorders and renal failure, cardiac disease, diabetic retinopathy and other ocular disorders, including blindness. A precursor to this condition, insulin resistance, may be a component in many age-related deteriorations and can result in alternating periods of both high and low blood sugar, uneven energy, obesity, hypertension and other disorders.

Diabetic conditions usually are treated medically in one of two ways. Insulin, the hormone which removes glucose from circulation, is supplied exogenously to treat the more severe cases in which the body's ability to produce this hormone is either impaired or nonexistent. Insulin therapy, therefore, is a treatment of last resort. Oral diabetes medications (such as sulphonylureas and biguanides) are also available. The drug metformin, a biguanide which is perhaps the safest and most successful of the usual oral hypoglycaemics, suppresses an elevated rate of basal hepatic glucose production, but has little benefit in extra-hepatic tissues and recent research has raised the possibility that its use by individuals who are not simultaneously using insulin may contribute to the development of Alzheimer's Disease. Insulin-sensitizing drugs offer broader benefits and are used to help slow the progression of diabetes, yet bring considerable side effects. Thiazolidinediones, including rosiglitazone and pioglitzone, are linked to a raised risk of fractures, thinning of the bones, weight gain and heart problems.

As taught in U.S. Pat. No. 6,207,714, an alternative treatment for blood glucose regulation is (−)-hydroxycitric acid (abbreviated herein as HCA), a naturally-occurring substance found chiefly in fruits of the species of Garcinia, and several synthetic derivatives of citric acid that have been investigated extensively with regard to their ability to inhibit the production of fatty acids from carbohydrates, to suppress appetite, and to inhibit weight gain. (Sullivan C, Triscari J. Metabolic regulation as a control for lipid disorders. I. Influence of (−)-hydroxycitrate on experimentally induced obesity in the rodent. Am J Clin Nutr. 1977 May;30(5):767-76.) Numerous other benefits have been attributed to the use of HCA, including, but not limited to an increase in the metabolism of fat stores for energy and an increase in thermogenesis. The commonly offered explanation for the effects of HCA is that this compound inhibits the actions of cytoplasmic (cytosolic) ATP:citrate lyase. (Clouatre D, Rosenbaum M E. The Diet and Health Benefits of HCA (Hydroxicitric Acid), 1994.) Weight loss benefits were attributed to HCA, its salts and its lactone in U.S. Pat. No. 3,764,692 granted to John M. Lowenstein in 1973. Lowenstein described a variety of possible pharmaceutical salts of HCA based upon alkali metals, e.g., potassium and sodium, and alkaline earth metals, e.g., calcium and magnesium. The exact mechanism by which HCA improves blood glucose metabolism has yet to be elucidated and the chief drawback with HCA treatment is the large dosage that often is required. Although, as taught in U.S. Pat. No. 6,207,714, there is a range of effective dosages, in practice the daily dosage usually is in the area of 5 grams of one of the HCA salts.

Free (−)-hydroxycitric acid, calcium, magnesium and potassium salts of HCA and poorly characterized mixtures of two or more of these minerals, usually substantially contaminated with sodium—and, sometimes, even free chloride ion with only the sodium has been removed—currently exist on the American market. Calcium/sodium salts have been sold widely since at least as early as 1992. Most of the HCA sold to date consists of calcium salts of varying degrees of purity and, more recently, of poorly characterized calcium and potassium mixtures. For instance, the currently best selling HCA salt (potassium-calcium hydroxycitrate) typically contains ≧10 percent impurities and the product specification allows for approximately 25 percent ±variations in the mg/gram of the potassium and calcium cations. (Shara M, Ohia S E, Schmidt R E, Yasmin T, et al. Physico-chemical properties of a novel (−)-hydroxycitric acid extract and its effect on body weight, selected organ weights, hepatic lipid peroxidation and DNA fragmentation, hematology and clinical chemistry, and histopathological changes over a period of 90 days. Mol Cell Biochem. 2004 May;260(1-2):171-86.) Safety issues have been raised with regard to the free acid and lactone forms of HCA due to their strong chelating properties and the risk of excessive loss of zinc from the body, a concern especially important to males both in puberty and in the later stages of life. The Inventors view properly manufactured potassium and potassium-magnesium salts as being the most desirable and efficacious choices.

As a result of the high dosages often required for efficacy using HCA salts alone to treat blood sugar abnormalities and lesser efficacy in subjects producing inadequate insulin, other approaches were sought to augment results. One was the use of bitter melon (Momordica charantia L.), plant extracts of which under certain conditions have exhibited significant benefits against both non-insulin dependent and insulin-dependent diabetes. Until now, however, the therapeutic use of the plant has proven to be highly problematic. Studies using powdered dried fruit have shown efficacy only at very high intakes, i.e., 15 grams or more per day. Standardized extracts such as are being sold in capsule form have proven to be ineffective. (Dans A M, Villarruz, M V, Jimeno C A, et al. The effect of Momordica charantia capsule preparation on glycemic control in type 2 diabetes mellitus needs further studies. J Clin Epidemiol 2007;60:554-559.) One product produced in Europe, Glukokine®, purports to have undergone two clinical trials (apparently Internet-published only) showing that one gram per day exhibits mild, yet significant efficacy in controlling blood sugar, but only after 6 months of use. Glucose tests were conducted only once every 6 weeks; using this measure, Glukokine would not have shown significance in the trial mentioned previously by Dans A M, et al. The studies were downloaded from http://www.vincoinc.com/i7:Web,Vinco,TechnicalInfo,V-GLK˜˜00000000.00000000.00000000.01010002.04112A88.14150A00.10530A16.3D8E00C7.

The employment in an animal model of compounds extracted with alcohol from the fruit demonstrated significant activity when these items were freshly prepared and given at high dosages. Freshly decocted fruit has been shown to be efficacious, yet the amount required typically is on the order of reduction from 100 grams of fresh fruit per day. The seeds contain a protein similar to bovine insulin and this insulin is effective, but typically must be given by injection or subcutaneously. Other issues have been raised with the daily intake of very large quantities of bitter melon. (Abascal K, Yarnell E. Using Bitter Melon to Treat Diabetes. Alternative & Complementary Therapies. 2005 August: 179-184.) Hence although bitter melon has shown promise for the regulation of blood sugar metabolism and diabetes, efficacy appears to demand freshly prepared material in relatively large amounts or extracts, again, in multi-gram amounts. Charantins, sapoinins commonly extracted and concentrated from the fruit for “standardized” preparations, appear to be completely inactive. (Harinantenaina L, Tanaka M, Takaoka S, Oda M, Mogami O, Uchida M, Asakawa Y. Momordica charantia constituents and antidiabetic screening of the isolated major compounds. Chem Pharm Bull (Tokyo). 2006 July;54(7):1017-21.)

Another item sometimes suggested for diabetes is sesame seed lignans. However, this recommendation is based on the impact of the lignans on hepatic fatty acid oxidation and serum triacylglycerol levels, not the impact on blood sugar metabolism. (Sirato-Yasumoto S, Katsuta M, Okuyama Y, Takahashi Y, Ide T. Effect of sesame seeds rich in sesamin and sesamolin on fatty acid oxidation in rat liver. J Agric Food Chem. 2001 May;49(5):2647-51.)

It is a surprising finding that the combination of these three items together leads to a dramatic synergy in which the level of intake of both HCA and bitter melon can be reduced to a fraction of that otherwise required. Although, as described above, both HCA and bitter melon are useful in the area of blood sugar metabolism and diabetes, and sesame lignans improve fatty acid oxidation, the dosages commonly required of the first two are quite high, and it remains unclear in the case of bitter melon whether under normal circumstances anything other than the fresh extract is successful.

Human trials with encapsulated bitter melon extract have failed. Sesame seed lignans on their own have not been proposed as improving serum glucose metabolism.

The synergy of this combination is all the more surprising in that combination products including HCA have been explored previously and the preferred dosage of HCA in such products remains on the order of equivalence to approximately 2,800 milligrams of (−)-hydroxycitric acid per day supplied by approximately 4,667 mg of an HCA salt in conjunction with 404 mg of other ingredients for a combination total of 5,071 mg. (U.S. Pat. Nos. 7,335,651; 7,153,877 and 7,119,110; also Preuss H G, et al. Effects of a natural extract of (−)-hydroxycitric acid (HCA-SX) and a combination of HCA-SX plus niacin-bound chromium and Gymnema sylvestre extract on weight loss. Diabetes Obes Metab. 2004 May;6(3):171-80.) In the preferred embodiment of the instant disclosure, only 1,200 mg of (−)-hydroxycitric acid equivalent in a total dosage volume of all ingredients of 2,800 mg is required as evidenced by experience with human subjects. The synergism of the instant disclosure is thus apparent in that the total dosage required for efficacy is less than one half of that taught in the previous art. (See Example 6.)

### SUMMARY

In one aspect the present disclosure is directed to a synergistic combination comprising (−)-hydroxycitric acid, bitter melon extract and sesame seed lignans extract.

In another aspect the present disclosure is directed to a pharmaceutical composition comprising synergistic combination of (−)-hydroxycitric acid, bitter melon extract and sesame seed lignans extract optionally along with acceptable additives.

In yet another aspect, the present disclosure is directed to a process for preparing synergistic combination comprising (−)-hydroxycitric acid, bitter melon extract and sesame seed lignans extract, said process comprising steps of: coating the (−)-hydroxycitric acid with controlled release agents; preparing the bitter melon and sesame seed lignans extracts; and mixing the bitter melon extract and the sesame seed lignans extract with the coated (−)-hydroxycitric acid to obtain the synergistic combination.

In still another aspect, the present disclosure is directed to a method of treating diabetes in a subject in need thereof, said method comprising step of administering pharmaceutically acceptable amount of synergistic combination comprising (−)-hydroxycitric acid, bitter melon extract and sesame seed lignans extract optionally along with pharmaceutically acceptable additives to the subject.

### DETAILED DESCRIPTION

The present disclosure is related to a synergistic combination comprising (−)-hydroxycitric acid, bitter melon extract and sesame seed lignans extract.

In another embodiment of the present disclosure, said (−)-hydroxycitric acid is selected from a group comprising potassium salt of (−)-hydroxycitric acid, sodium salt of (−)-hydroxycitric acid, magnesium salt of (−)-hydroxycitric acid, calcium salt of (−)-hydroxycitric acid, potassium-magnesium salt of (−)-hydroxycitric acid, potassium-calcium salt of (−)-hydroxycitric acid, sodium-calcium salt of (−)-hydroxycitric acid esters of (−)-hydroxycitric acid and amides of (−)-hydroxycitric acid, preferably potassium-magnesium salt of (−)-hydroxycitric acid.

In yet another embodiment of the present disclosure, said combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

In yet another embodiment of the present disclosure, preferred concentration of (−)-hydroxycitric acid is about 60% w/w, bitter melon extract is about 37.5% w/w and sesame seed lignans extract is about 2.5% w/w.

The present disclosure is related to a pharmaceutical composition comprising synergistic combination of (−)-hydroxycitric acid, bitter melon extract and sesame seed lignans extract optionally along with acceptable additives.

In another embodiment of the present disclosure, said synergistic combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

In yet another embodiment of the present disclosure, preferred concentration of (−)-hydroxycitric acid is about 60% w/w, bitter melon extract is about 37.5% w/w and sesame seed lignans extract is about 2.5% w/w.

In still another embodiment of the present disclosure, said additives are selected from a group comprising Vitamin B6, Vitamin B12, Biotin, Manganese, Chromium and Zinc.

In still another embodiment of the present disclosure, said pharmaceutical composition is formulated into dosage forms selected from a group comprising tablets, capsules, troches, lozenges, aqueous or oily suspensions, dispersible powders or granules, emulsion in hard or soft gel capsules, syrups and elixirs using additives selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

The present disclosure is related to a process for preparing synergistic combination comprising (−)-hydroxycitric acid, bitter melon extract and sesame seed lignans extract, said process comprising steps of: coating the (−)-hydroxycitric acid with controlled release agents; preparing the bitter melon and sesame seed lignans extracts; and mixing the bitter melon extract and the sesame seed lignans extract with the coated (−)-hydroxycitric acid to obtain the synergistic combination.

In yet another embodiment of the present disclosure, said controlled release agents are selected from a group comprising meltable coating agents, hydrolyzed vegetable oils, d-alpha-tocopheryl polyethylene glycol succinate (TPGS), polyethylene glycols (PEGs), polyethylene glycol glycerides composed of mono-, di- and triglycerides (gelucire), ono- and diesters of polyethylene glycol, and stearins of palm and palm fruit and combinations thereof.

In still another embodiment of the present disclosure, said (−)-hydroxycitric acid is selected from a group comprising potassium salt of (−)-hydroxycitric acid, sodium salt of (−)-hydroxycitric acid, magnesium salt of (−)-hydroxycitric acid, calcium salt of (−)-hydroxycitric acid, potassium-magnesium salt of (−)-hydroxycitric acid, potassium-calcium salt of (−)-hydroxycitric acid, sodium-calcium salt of (−)-hydroxycitric acid esters of (−)-hydroxycitric acid and amides of (−)-hydroxycitric acid, preferably potassium-magnesium salt of (−)-hydroxycitric acid and combinations thereof at a concentration ranging from about 1.0% to about 80% of the weight of the synergistic combination.

The present disclosure is related to a method of treating diabetes in a subject in need thereof, said method comprising step of administering pharmaceutically acceptable amount of synergistic combination comprising (−)-hydroxycitric acid, bitter melon extract and sesame seed lignans extract optionally along with pharmaceutically acceptable additives to the subject.

In yet another embodiment of the present disclosure, said synergistic combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

In still another embodiment of the present disclosure, preferred concentration of (−)-hydroxycitric acid is about 60% w/w, bitter melon extract is about 37.5% w/w and sesame seed lignans extract is about 2.5% w/w.

In still another embodiment of the present disclosure, the subject is an animal or human being.

In still another embodiment of the present disclosure, said method reduces reactive hypoglycemia in subjects resistant to insulin.

In still another embodiment of the present disclosure, the synergistic combination comprising (−)-hydroxycitric acid dose of about 1,200 mg, bitter melon extract of about 750 mg and sesame seed lignans extract of about 50 mg.

In this disclosure it was found that the glucose and insulin metabolism in an individual showing evidence of dysregulation, as is found in insulin resistance and diabetes, is improved when that person receives an appropriate oral administration of the combination of (−)-hydroxycitric acid, bitter melon extract and sesame seed lignans. In the appropriate form(s) and amounts, the combination acts as a hypoglycemic agent that is far more efficacious than the sum of the effects of the individual ingredients. It also reduces reactive hypoglycemia in those who are insulin resistant. The potassium and potassium-magnesium salts of (−)-hydroxycitric acid are the preferred form of the compound, followed by the sodium salt.

Various salts of (−)-hydroxycitric acid (calcium, magnesium, potassium, sodium and mixtures of these) have been available commercially for several years. Any of these materials can be used to fulfill the disclosure revealed here, but with varying degrees of success. These materials are generally useful in this descending order of efficacy: potassium salt or potassium-magnesium salt, sodium salt, potassium-calcium salt, magnesium salt and the calcium salt. Experimental data, however, suggests that product purity and manufacturing process plays a key role in product efficacy; for example, some researchers have found commercially available potassium-calcium salts of HCA to have little or no effect on blood sugar levels at any of the normally suggested dosage levels. The previously patented hydroxycitric acid derivatives (mostly amides and esters of hydroxycitric acid, the patents for which are now expired), in the absence of actual clinical data, are presumed to be roughly equivalent to the HCA sodium salt in efficacy. The pure potassium and sodium salts, in practice, are less desirable due either to the sodium content or to major difficulties in handling because of their high pH and hygroscopic nature. Attempts at overcoming the latter two drawbacks have been of mixed success as some animal trials indicate much-reduced uptake as a result.

The bitter melon extract should not be limited to or standardized on charantins, which appear to be compounds with low biological activity. Rather, the extract may be produced in a variety of ways, such as expressing and then drying the fresh juice to powder, seriatim water and then alcohol extraction to produce powders, and or the seriatim employment of other solvents to gather the full range of constituents present, then subsequently reduced to powder.

Sesame seed lignan extracts preferably will contain 10-90 percent sesamin. Mixtures of sesamin, sesamolin and other lignans are commercially available.

The preferred ratio of components with the HCA salt as its (−)-hydroxycitric acid equivalent is in the range of 50-70 percent HCA, 27.5-47.5 percent bitter melon extract and 1.5-3.5 percent sesame seed lignans (as a 30% sesamin extact), with the most desirable ratio being 60 percent HCA, 37.5 percent bitter melon extract and 2.5 percent sesame seed lignans (as a 30% sesamin extract). Maintenance dosing has been successful with as little as one tablet from Example 2 taken per day and supplying 800 mg of (−)-hydroxycitric acid equivalent, 375 mg bitter melon extract and 25 mg sesame seed lignans (as a 30% sesamin extract). Most test subjects have been successful with one tablet taken twice per day an hour before meals, hence supplying 1,200 mg (−)-hydroxycitric acid equivalent, 750 mg bitter melon extract and 50 mg sesame seed lignans (as a 30% sesamin extract). Very large individuals and those with muted insulin release may require 4 tablets per day taken as 2 tablets ingested twice per day an hour before meals and supplying 2,400 mg (−)-hydroxycitric acid equivalents, 1,500 mg bitter melon extract and 100 mg sesame seed lignans (as a 30% sesamin extract).

### EXAMPLE 1

### HCA, Bitter Melon and Sesame Seed Lignan Sachets and Capsules

TABLE 1
Ingredients along with their concentrations

| Item No. | Ingredient | Active Mg/4 sachets |
|---|---|---|
| 1 | KMg (−)-Hydroxycitrate- | 4,000.00 (equivalent to 2.400 mg |
| | 60% | HCA as free acid) |
| 2 | Bitter Melon Extract | 1,500.00 |
| 3 | Sesame Seed Lignans | 100.00 (30 mg sesamin) |
| | (30% sesamin) | |
| | Total | 5,600.00 mg |

This formula provided in table 1, contains only the core ingredients with the HCA in the form of potassium-magnesium hydroxycitrate. It can be mixed and placed in sachets or capsules as is, or stamped into tablets with the help of the necessary excipients and then a coating applied to the tablets by standard procedures. A typical dose with this formula is one sachet in water twice per day one hour before meals. Very large individuals or those with muted insulin production may benefit from two sachets taken twice per day one hour before meals or as directed by the attending physician. The limits of this formulation are a gradual interaction of the HCA with the other two ingredients over time and moisture intrusion leading to degradation in capsules, hence short shelf life. In its most active salts (potassium, potassium-magnesium and sodium), HCA is hygroscopic and it can bind to gums, fiber, catechins and various other ingredients. For this reason, special coatings have been developed. See Examples 2 and 3 for procedures to overcome these limitations.

### EXAMPLE 2

### HCA (Coated), Bitter Melon and Sesame Seed Lignan Tablets

TABLE 2
Ingredients along with their concentrations

| Item No. | Ingredient | Active Mg/4 tab |
|---|---|---|
| 1 | KMg (−)-Hydroxycitrate- | 4,000.00 (equivalent to 2.400 mg |
| | 60% coated @ 20% | HCA as free acid) |
| 2 | Bitter Melon Extract | 1,500.00 |
| 3 | Sesame Seed Lignans | 100.00 (30 mg sesamin) |
| | (30% sesamin) | |
| | Total | 5,600.00 mg |

This formula provided in table 2, contains only the core ingredients with the HCA in the form of potassium-magnesium hydroxycitrate, but coated to prevent interaction with the bitter melon extract and the sesame seed lignans as well as to increase capsule and tablet life. The coating preferably is performed as taught in US Patent Application 20060141030. Other acceptable coating procedures are taught in U.S. Pat. Nos. 6,207,714 and 7,189,416; also US Patent Application 20060292216. The formula can be mixed and placed in sachets and capsules as is or stamped into tablets with the help of the necessary excepients and then a coating applied to the tablets by standard procedures. A typical dose with this formula is one tablet twice per day one hour before meals. Very large individuals or those with muted insulin production may benefit from two tablets taken twice per day one hour before meals or as directed by the attending physician. Maintenance dosing has been successful with as little as one tablet.

### EXAMPLE 3

### HCA (Coated), Bitter Melon and Sesame Seed Lignan Plus Other Nutrients Tablets

TABLE 3
Ingredients along with their concentrations

| | | | | Raw Material | | | Raw | RM |
|---|---|---|---|---|---|---|---|---|
| Item | | | | Mg/4 | | | Material | Actual |
| No. | Ingredient | Active Mg/4 tab | | tab | | Coefficient | Actives | per kg |
| 1 | Vitamin B6 HCl | 15.00 | | 18.0722 | | 0.83 | 0.2218 | 0.2673 |
| 2 | B12 | 0.05 | | 5.0 | | 0.01 | 0.00739 | 0.0739 |
| | (methylcobalamin) | | | | | | | |
| | 1.0% in DCP | | | | | | | |
| 3 | Biotin | 0.40 | | 0.4 | | 1 | 0.0059 | 0.0059 |
| 4 | Manganese | 1.00 | | 2.0 | | 0.5 | 0.0147 | 0.0295 |
| | (citrate | | | | | | | |
| 5 | Chromium Yeast | 0.15 | | 75 | | .002 | 0.0022 | 1.1093 |
| | @ 2 mcg/mg | | | | | | | |
| 6 | Zinc | 15.00 | | 60 | | 0.25 | 0.2218 | 0.8874 |
| | (monomethionine) | | | | | | | |
| 7 | KMg (−)- | 4,000.00 | | 5000 | | 0.8 | 59.1639 | 73.9549 |
| | Hydroxycitrate - | (2.400 | mg | | | | | |
| | 60% coated @ | | free acid) | | | | | |
| | 20% | | | | | | | |
| 8 | Bitter Melon | 1,500.00 | | 1500 | | 1 | 22.1864 | 22.1864 |
| | extract | | | | | | | |
| 9 | Sesame Seed | 100.00 | | 100 | | 1 | 1.4790 | 1.4790 |
| | Lignans (30% | (30 | mg | (30 | mg | | | |
| | sesamin) | | sesamin) | | sesamin) | | | |
| 10 | Folic Acid (folate) | 0.40 | | 0.4 | | 1 | 0.0059 | 0.0059 |
| | Total | 5,632.00 | mg | 6760.8722 | | | 83.302339 | 99.9995 |

This is a more elaborate formula provided in table 3, designed to address other issues typically found with diabetics, for instance, deficiencies in vitamin B6 and greater needs for folic acid for cardiovascular protection. As with Example 2, this formula can easily be blended and encapsulated directly or, using known procedures, can be stamped into tablets and coated. It should be noted that pyridoxine HCl is used, hence the coefficient is 0.83 for B6 activity; and methycobalamin requires dilution 100 times for proper distribution giving al.0% triturate in DCP, hence the coefficient is 0.01. Dosage is as in Example 2.

### EXAMPLE 4

### Pilot Animal Stretozotocin-Induced Diabetes Trial

The synergism of the formula is reflected in findings in an animal trial of induced diabetes treated with Metformin. To produce streptozotocin (STZ)-induced diabetes in experimental rats, male Wistar albino rats of 12 weeks age were allowed to fast 24 hrs prior to injection with freshly prepared STZ in citrate buffer [pH7.4] (45 mg/kg, i.p). After one week, rats with marked hyperglycemia (fasting blood glucose>300) were used for the planned study. However, an additional arm of similarly-induced diabetic rats that were older and much sicker was added to test the herbal mixture prepared to produce the relative percentages of each ingredient as in Example 1 given above, here termed “STD Herbal.”

After an overnight fast, the samples suspended in 5% gum acacia were fed by gastric intubations with a syringe. Blood samples were collected for the measurement of blood glucose from the tail vein at 0, 1, 2, 3 & 4 hrs. The blood glucose level was determined by using an electronic glucometer (Accu Check, Roche Diagnostic) and the results are tabulated in table 4.

TABLE 4
Blood glucose levels in albino rats

| | Dose | Initial | Treatment | | | |
|---|---|---|---|---|---|---|
| Treatment | (mg/kg) | (0 hr) | +1 hr | +2 hr | +3 hr | +4 hr |
| STD | 250 | 429.16 ± 26.59 | 390.5 ± 28.78 | 330.66 ± 40.58* | 286.66 ± 37.31* | 197.83 ± 25.50* |
| Herbal | | | (9.0) | (22.95) | (33.20) | (53.90) |
| Metfor | 150 | 329.2 ± 5.25 | 271.6 ± 4.0 | 214.4 ± 3.17* | 176 ± 6.7* | 153 ± 3.31* |
| min | | | (17.49) | (34.87) | (46.38) | (53.52) |

n=6 animals in each group. Parenthesis value indicate percentage reduction of blood sugar level; *P<0.01 compared with initial level of blood glucose (0 h) in the respective group. Data were analyzed by repeated measure ANOVA followed by Dunnett test. The results shown are quite impressive and indicate efficacy in a range equal to or greater than that found with Metformin, a standard drug used to treat diabetes. The impact of STD Herbal was slower in onset, but was more progressive, suggesting that a further extended time frame might have shown even better results.

### EXAMPLE 5

### Clinical Experience with Diabetics

Each of the first three of the following case studies utilized the formula described in Example 2 at the rate of one tablet twice per day for a total of 2,800 mg combined active ingredients. The fourth subject, who was quite heavy at more than 250 pounds, used 2 tablets twice per day.

Female (Chinese, 89 years old). An immobilized stroke victim being fed a standard medical liquid diet manufactured in Canada via a tube. Initially, her fasting blood sugar levels were consistently above 200. She had been drifting in and out of coma for six months. Her doctor had indicated that she needed to begin insulin therapy, but her family had resisted. The attending nurses (24 hour in-room observation) would grind up one tablet twice a day and feed with water via her feeding tube. By Day 3 her fasting glucose levels had begun significant decline. On Day 5 her fasting blood sugar levels entered the 140-150 range and she became fully conscious for the first time in several months. Following three months of treatment, her fasting levels stabilized at 120 or slightly below. Her doctor concluded that the patient, then 90, did not require additional medication.

Male (Chinese, 85 years old). Type 2 diabetic (not well controlled), approximately 55 kg in weight, injecting insulin and taking high blood pressure medications (diuretic). He began taking one tablet twice a day. Initially, he began his first dose before breakfast, but complained of minor stomach discomfort so the regimen was changed to a first dose prior to lunch. By Day 3, he reported that his blood pressure had begun to decline and that his blood sugar levels were more stable and better controlled by his insulin regimen. He subsequently began gradually reducing his blood pressure medication and over a period of 3-4 months completely stopped taking it. During this time, he also began reducing his insulin use and found that he could maintain fasting blood sugar levels at around 150 without the use on any insulin injections. Due to his aversion to the injections, he no longer uses insulin.

Subject consumed a relatively low-fat diet based on white rice as the carbohydrate staple with fish and chicken as the protein sources and adequate vegetable intake.

Female (Caucasian, 62 years old). A woman 62 years of age, 5′11″ tall, approximately 165 pounds in weight and otherwise in apparently excellent health, experienced fasting blood glucose levels of 125-130 and postprandial levels of 150-160. She began taking one tablet prior to breakfast and another before dinner. She reported that her postprandial blood sugar levels declined from the 150 range to below 100 by Day 4. She volunteered how much better she felt and slept. Subject consumed a relatively low-fat diet based on white rice as the carbohydrate staple with fish and chicken as the protein sources and adequate vegetable intake.

Man (Caucasian, 50 years old and weighing more than 250 pounds). Subject was an obese pre-diabetic suffering from long-standing moderately high blood pressure in the 140/90 range, but not using any blood pressure medication. Initial dosage was 2 tablets twice per day. By Day 10, his blood pressure had dropped to the 120/70 range. He also reported much improved sleep and better energy levels. In addition, his desire for constant snacking, including prior to sleep, was attenuated. Subject had been borderline in serum glucose, LDL/HDL and triglycerides, all of which improved to within the “normal” range by day 10.

### EXAMPLE 6

### HCA and Bitter Melon requirements as Single Ingredients

Of the three ingredients of the present disclosure, sesame lignans have not been proposed as having significant effects on either blood glucose or insulin metabolism. Sesame oil used as a substitute for other oils in the diet has demonstrated benefits in these areas; however, the lignans do not affect the serum glucose concentrations. (Kushiro M, et al. Comparative effect of sesamin and episesamin on the activity and gene expression of enzymes in fatty acid oxidation and synthesis in rat liver. J Nutr Biochem. 2002 May;13(5):289-295.) Hence, sesame lignans need not be further addressed except as a component of the disclosure.

In the present disclosure, efficacy in humans has been discovered with only 1,200 mg of (−)-hydroxycitric acid equivalent derived from the potassium-magnesium salt and 750 mg bitter melon extract plus sesame 50 mg sesame lignans for a 2,800 mg total weight of HCA salt plus herbs. In contrast, if HCA salt (measured as free acid) or bitter melon extract alone is used, the amount of either is much higher. In published literature, the lowest dose of HCA as free acid equivalent found efficacious in reducing blood glucose is 3 grams derived from the potassium salt (U.S. Pat. No. 6,207,714). A smaller dosage of a potassium salt (equivalent to 2,170 mg HCA free acid) proved to be ineffective, just as 3 grams HCA as the free acid equivalent derived from the calcium salt proved to be ineffective. Using the potassium-calcium salt, the lowest stand-alone dose that has been shown to be effective is the equivalent of 3,276 mg of HCA free acid, which is more than 30% greater than the total dosage of all components measured as bulk rather than actives of the present disclosure.

Even when combined with putatively synergistic components as in U.S. Pat. Nos. 7,335,651; 7,153,877 and 7,119,110, the HCA free acid equivalent of the potassium-calcium salt is 2,800 mg and the total blend weight is 5,071 mg. The difference thus is 1,200 mg HCA as free acid equivalent with the disclosure versus 3,276 mg HCA as free acid equivalent, an amount significantly greater than the total weight of the efficacious dose of all ingredients of the disclosure taken together. Hence, the present Disclosure is profoundly more active than the previous art.

The question then becomes whether prior art teaches that a small amount of dried bitter melon extract added to the HCA of the current disclosure can account for the improved efficacy on a merely additive basis. Prior art teaches that this is not possible as an additive effect. A recent review monograph indicates that the dosage for encapsulated dried powder is 3-15 grams per day. (Momordica charantia (bitter melon). Monograph. Altern Med Rev. 2007 December;12(4):360-3.) A double-blinded clinical trial of a proprietary bitter melon extract delivered via capsules achieved a null effect despite delivering 3 grams of the extract per day. ((Dans A M, Villarruz, M V, Jimeno C A, et al. The effect of Momordica charantia capsule preparation on glycemic control in type 2 diabetes mellitus needs further studies. J Clin Epidemiol 2007;60:554-559.) Even special extracts of the vegetable may require as much as the equivalent dosage of 7 grams in a 70 kg human. (Uebanso T, et al. Extracts of Momordica charantia suppress postprandial hyperglycemia in rats. J Nutr Sci Vitaminol (Tokyo). 2007 December;53(6):482-8.) Only the freshly expressed juice of the bitter melon consumed immediately has shown acceptable efficacy at low dosage levels.

Therefore, inasmuch as the effective dosage of dried bitter melon extracts is known to be in the range of 3-15 grams, it is not possible that the efficacy of the current disclosure can rest on an additive benefit of bitter melon extract in conjunction with an HCA salt.

The following table 6 employs a 6:1 multiplier when translating dosages used with rats to those appropriate for humans. This is a standard recently affirmed that reduces the quantitative extrapolation from the animal model to human equivalents. (Reagan-Shaw S, Nihal M, Ahmad N. Dose translation from animal to human studies revisited. FASEB J. 2008 Mar;22(3):659-61.) Older literature used a 5:1 multiplier. (Freireich E J, et al. Quantitative comparison of toxicity of anticancer agents in mouse, rat, hamster, dog, monkey, and man. Cancer Chemother Rep. 1966 May;50(4):219-44.) The novelty of the present disclosure is even more pronounced if the older standard is used inasmuch as the extrapolated doses of HCA salts required to show efficacy would be much higher. Most data based on HCA salts added as a percentage of the diet as opposed to being quantified separately has been excluded due to obvious difficulties in establishing equivalencies. Similarly, mouse data has been excluded because the mouse is unpredictably and exceedingly receptive to the actions of (−)-hydroxycitrate in ways that skew quantitative comparisons.

TABLE 6
Comparison of dosage requirements for instant disclosure vis-à-vis the known
literature

| | | Dosage | |
|---|---|---|---|
| | Model and HCA | (Salt amount, then | |
| Study | Salt Type | equivalents) | Results |
| Disclosure Example 4 | Human diabetics/ | 2,000 mg salt | Significantly |
| | potassium- | yielding 1,200 mg | reduced plasma |
| | magnesium salt plus | HCA free acid | glucose |
| | bitter melon extract | equivalent plus 750 mg | |
| | and sesame lignans | bitter melon | |
| | (Glykon) | extract and 50 mg | |
| | | sesame lignans from | |
| | | 100 mg of a 30% | |
| | | sesamin preparation = | |
| | | 2,800 mg total | |
| | | weight of | |
| | | ingredients | |
| Preuss HG, et al. | Human obese | 4,667 mg salt | Proposed for |
| Effects of a natural | subjects/ | yielding 2,800 mg | Metabolic |
| extract of (−)- | potassium-calcium | HCA free acid | Syndrome; |
| hydroxycitric acid | salt plus chromium | equivalent plus 400 mg | demonstrated |
| (HCA-SX) and a | and Gymnema | Gymnema | improved oxidation |
| combination of | sylvestre extract | extract and 4 mg | of fats for energy; |
| HCA-SX plus | (Interhealth) | niacin-bound | however, there is no |
| niacin-bound | | chromium = 5,071 mg | evidence or claim |
| chromium and | | total weight of | that this |
| Gymnema sylvestre | | ingredients | combination |
| extract on weight | | | improves plasma |
| loss. Diabetes Obes | | | glucose or insulin |
| Metab. 2004 | | | metabolism in either |
| May; 6(3): 171-80; | | | humans or animals |
| ingredient(s) in U.S. | | | at this level of |
| Pat. Nos. 7,335,651; | | | intake. |
| 7,153,877 and | | | |
| 7,119,110. | | | |
| Gatta B, | Human (males, | 4, 000 mg of 50% | No effect on plasma |
| Zuberbuehler C, et | approx. 23 years | material yielding 2,000 mg | glucose or insulin |
| al. Acute effects of | old)/calcium salt | as HCA | |
| pharmacological | (Sabinsa) | free acid | |
| modifications of | | equivalent | |
| fatty acid | | delivered via 3 | |
| metabolism on | | doses | |
| human satiety. Br J | | | |
| Nutr. 2008 Dec 16: 1-11. | | | |
| Super CitriMax | Obese Zucker rat/ | 500 mg/kg body | Significantly |
| (HCA-SX) | potassium-calcium | weight/day for 2 wks | reduced plasma |
| attenuates increases | salt (Interhealth) | increased to | glucose and insulin. |
| in oxidative stress, | | 1,500 mg/kg body | |
| inflammation, | | weight for | |
| insulin resistance, | | additional 5 wks = | |
| and body weight in | | 70 kg human | |
| developing obese | | dosage of 3,500 mg | |
| Zucker rats. Asghar M, | | increased to 10,500 mg | |
| et al. Mol Cell | | as HCA free | |
| Biochem. 2007 | | acid equivalent | |
| Oct; 304(1-2): 93-9. | | | |
| Leonhardt M, | Male Sprague- | 60% HCA salt | Oral glucose |
| Balkan B, Langhans | Dawley rat/ | added to food for | tolerance test |
| W. Effect of | potassium-calcium | 3% of diet = | showed |
| hydroxycitrate on | salt (Interhealth) | approx. 350 mg/kg | significantly |
| respiratory quotient, | | body weight/day = | decreased increase |
| energy expenditure, | | 70 kg human | in plasma glucose |
| and glucose | | dosage of 4,083 mg | from baseline (Δ |
| tolerance in male | | as HCA free acid | glucose), trend to |
| rats after a period of | | equivalent | decreased area |
| restrictive feeding. | | | under the curve for |
| Nutrition. 2004 | | | glucose; however, Δ |
| Oct; 20(10): 911-5. | | | insulin and area |
| | | | under the curve for |
| | | | insulin did not differ |
| | | | between groups. |
| Effects of niacin- | Aged diabetic obese | 234 mg/kg body | Significantly |
| bound chromium, | Zucker rat/ | weight/day for 3 wks | reduced plasma |
| Maitake mushroom | potassium-calcium | increased to | glucose. |
| fraction SX and (−)- | salt (Interhealth) | 468 mg/kg body | |
| hydroxycitric acid on | | weight for 3 wks = | |
| the metabolic | | 70 kg human | |
| syndrome in aged | | dosage of 1,638 mg | |
| diabetic Zucker fatty | | increased to 3,276 mg | |
| rats. Talpur N, et al. | | as HCA free | |
| Mol Cell Biochem. | | acid equivalent | |
| 2003 Oct; 252(1-2): | | | |
| 369-77. | | | |
| Hydroxycitrate | Male Wistar rats/ | 310 mg/kg body | Reduced insulin |
| (HCA) reduces the | potassium salt | weight acutely = 70 kg | response to an |
| insulin response to | (Regulator) | human dosage of | intragastric glucose |
| an intragastric | | 2,170 mg as HCA | load, but no |
| glucose load. | | free acid | significant effect on |
| Wielinga PY, et al. | | equivalent | insulin or glucose |
| Appetite | | | with an intravenous |
| 2003; 40:369. | | | glucose tolerance |
| Abstract | | | test. |
| Effects of acute (−)- | Human - trained | 18 grams as HCA | No significant effect |
| hydroxycitrate | athletes/calcium | free acid | on plasma glucose. |
| supplementation on | salt (Interhealth) | equivalent | |
| substrate metabolism | | | |
| at rest and during | | | |
| exercise in humans. | | | |
| van Loon LJ, et al. | | | |
| Am J Clin Nutr. | | | |
| 2000 | | | |
| Dec; 72(6): 1445-50. | | | |
| (−)-Hydroxycitric | Human - sedentary/ | 3,000 mg as HCA | No significant effect |
| acid does not affect | calcium salt | free acid | on plasma glucose |
| energy expenditure | (Sabinsa) | equivalent | or insulin. |
| and substrate | | | |
| oxidation in adult | | | |
| males in a post- | | | |
| absorptive state. | | | |
| Kriketos AD, et al. | | | |
| Int J Obes Relat | | | |
| Metab Disord. 1999 | | | |
| Aug; 23(8): 867-73. | | | |
| U.S. Pat. No. | Human morbidly | 5 grams KHCA = | Significantly |
| 6,207,714 | obese diabetics/ | 70 kg human | reduced plasma |
| Methods and | potassium salt | dosage of 3,000 mg | glucose. |
| pharmaceutical | (Glykon) | HCA free acid | |
| preparations for | | equivalent, but | |
| improving glucose | | subjects in this pilot | |
| metabolism with (−)- | | trial averaged | |
| hydroxycitric acid. | | approx. 150 kg | |
| (2001) | | | |

What is claimed is:

1) A synergistic combination comprising (−)-hydroxycitric acid, bitter melon extract and sesame seed lignans extract.

2) The combination as claimed in claim 1, wherein said (−)-hydroxycitric acid is selected from a group comprising potassium salt of (−)-hydroxycitric acid, sodium salt of (−)-hydroxycitric acid, magnesium salt of (−)-hydroxycitric acid, calcium salt of (−)-hydroxycitric acid, potassium-magnesium salt of (−)-hydroxycitric acid, potassium-calcium salt of (−)-hydroxycitric acid, sodium-calcium salt of (−)-hydroxycitric acid esters of (−)-hydroxycitric acid and amides of (−)-hydroxycitric acid, preferably potassium-magnesium salt of (−)-hydroxycitric acid.

3) The combination as claimed in claim 1, wherein said combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

4) The combination as claimed in claim 3, wherein preferred concentration of (−)-hydroxycitric acid is about 60% w/w, bitter melon extract is about 37.5% w/w and sesame seed lignans extract is about 2.5% w/w.

5) A pharmaceutical composition comprising synergistic combination of (−)-hydroxycitric acid, bitter melon extract and sesame seed lignans extract optionally along with acceptable additives.

6) The composition as claimed in claim 5, wherein said synergistic combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

7) The composition as claimed in claim 6, wherein preferred concentration of (−)-hydroxycitric acid is about 60% w/w, bitter melon extract is about 37.5% w/w and sesame seed lignans extract is about 2.5% w/w.

8) The composition as claimed in claim 5, wherein said additives are selected from a group comprising Vitamin B6, Vitamin B12, Biotin, Manganese, Chromium and Zinc.

9) The composition as claimed in claim 5, wherein said pharmaceutical composition is formulated into dosage forms selected from a group comprising tablets, capsules, troches, lozenges, aqueous or oily suspensions, dispersible powders or granules, emulsion in hard or soft gel capsules, syrups and elixirs using additives selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

10) A process for preparing synergistic combination comprising (−)-hydroxycitric acid, bitter melon extract and sesame seed lignans extract, said process comprising steps of:
(a) coating the (−)-hydroxycitric acid with controlled release agents;
(b) preparing the bitter melon and sesame seed lignans extracts; and
(c) mixing the bitter melon extract and the sesame seed lignans extract with the coated (−)-hydroxycitric acid to obtain the synergistic combination.

11) The process as claimed in claim 10, wherein said controlled release agents are selected from a group comprising meltable coating agents, hydrolyzed vegetable oils, d-alpha-tocopheryl polyethylene glycol succinate (TPGS), polyethylene glycols (PEGs), polyethylene glycol glycerides composed of mono-, di- and triglycerides (gelucire), ono- and diesters of polyethylene glycol, and stearins of palm and palm fruit and combinations thereof.

12) The process as claimed in claim 10, wherein said (−)-hydroxycitric acid is selected from a group comprising potassium salt of (−)-hydroxycitric acid, sodium salt of (−)-hydroxycitric acid, magnesium salt of (−)-hydroxycitric acid, calcium salt of (−)-hydroxycitric acid, potassium-magnesium salt of (−)-hydroxycitric acid, potassium-calcium salt of (−)-hydroxycitric acid, sodium-calcium salt of (−)-hydroxycitric acid esters of (−)-hydroxycitric acid and amides of (−)-hydroxycitric acid, preferably potassium-magnesium salt of (−)-hydroxycitric acid and combinations thereof at a concentration ranging from about 1.0% to about 80% of the weight of the synergistic combination.

13) A method of treating diabetes in a subject in need thereof, said method comprising step of administering pharmaceutically acceptable amount of synergistic combination comprising (−)-hydroxycitric acid, bitter melon extract and sesame seed lignans extract optionally along with pharmaceutically acceptable additives to the subject.

14) The method as claimed in claim 13, wherein said synergistic combination comprises (−)-hydroxycitric acid at a concentration ranging from about 50% w/w to about 70% w/w, bitter melon extract at a concentration ranging from about 27.5% w/w to about 47.5% w/w and sesame seed lignans extract at a concentration ranging from about 1.5% w/w to about 3.5% w/w.

15) The method as claimed in claim 14, wherein preferred concentration of (−)-hydroxycitric acid is about 60% w/w, bitter melon extract is about 37.5% w/w and sesame seed lignans extract is about 2.5% w/w.

16) The method as claimed in claim 13, wherein the subject is an animal or human being.

17) The method as claimed in claim 13, wherein said method reduces reactive hypoglycemia in subjects resistant to insulin.

18) The method as claimed in claim 13, wherein the synergistic combination comprising (−)-hydroxycitric acid dose of about 1,200 mg, bitter melon extract of about 750 mg and sesame seed lignans extract of about 50 mg.
